(19) United States
(12) Patent Application Publication
(10) Pub. No.: US 2022/0270520 A1
(43) Pub. Date: Aug. 25, 2022

(54) FLEXIBLE SELF-ADHESIVE LABEL COMPRISING A RADIATION-SENSITIVE INDICATOR FOR A SYRINGE

(71) Applicant: ISP Investments LLC, Wilmington, DE (US)

(72) Inventors: Robert CROHN, Towaco, NJ (US); David K. HOOD, Basking Ridge, NJ (US); Emily Jean DREW, Flemington, NJ (US); Maria MENCHON, Bridgewater, NJ (US)

(73) Assignee: ISP Investments LLC, Wilmington, DE (US)

(21) Appl. No.: 17/628,024

(22) PCT Filed: Jul. 20, 2020
(86) PCT No.: PCT/US2020/042792

§ 371 Date: Jan. 18, 2022

Related U.S. Application Data
(60) Provisional application No. 62/875,804, filed on Jul. 18, 2019.

(51) Int. Cl. G09F 3/00; A61M 5/315; A61L 2/28

(52) U.S. Cl. CPC G09F 3/0291; A61M 5/315; A61L 2/28; G09F 3/0297; A61M 2005/3125

(57) ABSTRACT

A flexible self-adhesive label comprising a radiation-sensitive indicator for a syringe, the syringe comprising a barrel part with a front part and a rear part, having a tip at the front part, a piston at the rear part, and an exterior surface with a marking thereon. The label is attached to at least one part of the syringe and does not overlap with the marking. Also disclosed is a labeling method, kit and dispenser featuring such labels.

### FIELD OF THE INVENTION

The presently disclosed process(es), procedure(s), method(s), product(s), result(s), and/or concept(s) (collectively referred to hereinafter as the “present disclosure or invention”) relates generally to a flexible self-adhesive label comprising a radiation-sensitive indicator for a blood containing product such as a syringe, and to methods, kits and dispensers featuring such flexible self-adhesive labels.

### BACKGROUND OF THE INVENTION

The use of dosimeters to determine the absorbed dose of ionizing radiation received by the person or the substrate to which it is attached is well known. There are many types of dosimeters, the most common being the air-capacitor dosimeter, the film badge and the thermoluminescent dosimeter.

The air-capacitor dosimeter includes an inside chamber which serves as an ion chamber, and a central collecting electrode. An electric charge is placed on the central electrode. Such dosimeters, when charged, are essentially air-capacitors, and the amount of discharge during use is proportional to the absorbed dose of X-ray or gamma ray radiation received. Inherent in the use of such dosimeters is the need of a power source for applying a voltage between the electrode and the chamber wall.

A film badge dosimeter usually is loaded with one or more film packets. The simplest type of film badge consists of a small paper envelope containing a film, such as a dental film, one-half of which is surrounded by a thin lead foil. The badge must contain one or more filters so that a comparison can be made of the relative blackening of the developed film from behind the various filters. This comparison reveals the extent of exposure to various types of radiation. Inherent in the use of such badge is the need of a developing process to develop the exposed film.

A thermoluminescent dosimeter is one that functions on the principle of thermoluminescence; that is, the property of certain substances that release light upon heating after they have been exposed to ionizing radiation. Inherent in this type of dosimeter is the need for measuring either the peak intensity or the integrated quantity of the light emitted.

While such dosimeters perform satisfactorily, they require outside equipment or processes in order to function and/or be readable. That is, one cannot detect merely by visually observing the exposed material, the level of absorbed radiation. For this reason, various self-developing dosimeters have achieved widespread commercial acceptance.

In U.S. Pat. No. 4,001,587 to Georgy Mitrofanovich Panchenkov et al dated Jan. 4, 1977, dosimeters are disclosed which incorporate various dyes, some acid-sensitive and some not acid-sensitive, which change their color on exposure to radiation.

U.S. Pat. Nos. 5,051,597 and 5,084,623 disclose a radiation dosage indicator having a radiation sensitive zone capable of changing opacity in response to exposure to radiation to change the visibility of indicia on said indicator, and to the method of manufacturing such indicator.

U.S. Publication No. 2016/0290859 discloses a film that is specifically manufactured for measuring long wavelength Ultraviolet (UVA) light. More specifically, it relates to a film manufactured for use in indicating an exposure and/or measuring dose of the exposure of long wavelength UV that is commonly used for UV curing of coating, pathogen inactivation and other industrial and medical applications.

U.S. Design Pat. No. 458,642 discloses an ornamental design for a radiation indicator tag.

U.S. Pat. No. 4,536,450 discloses a nonlinear optical, piezoelectric, pyroelectric, waveguide.

U.S. Pat. No. 7,445,880 discloses photochromic filaments composed of the lithium salt of a conjugated, polymerizable polyacetylene having a carboxylic acid or carboxylate terminal group.

There are several important parameters to consider once a blood product is put into a syringe. For example, transfusionists might transfer aliquots of the desired units into a syringe at the patient's bedside so that the transfusion rate and volume can be controlled. Also, storage in syringes for up to 6 hours results in platelets that are generally acceptable by FDA standards and that appear acceptable after passage through the syringe. However, it is advisable to avoid storage at 37° C. and to minimize storage times and storage of volume-reduced platelets.

Syringes are among the most problematic devices in a hospital environment, often causing cuts or needle sticks on healthcare workers. It would be highly beneficial to improve the process flow in this environment and reduce safety hazards and risks. Hospital processing departments are moving towards or considering irradiation of blood products in syringes for neonatal and pediatric patient populations. With the advent of new X-ray irradiators designed to irradiate syringes, irradiated syringes allow hospitals to better safely manage and handle their blood products. In syringe applications, such as during blood transfusions, a safe, quick and clear distinction is very important to determine whether the blood has been sterilized or not.

In syringe applications, where blood product is required for neonatal and pediatric transitions, smaller syringes are generally employed. Current indicators are unacceptable due in part to their size interfering with the healthcare workers ability to see the markings on the syringe. This results in unacceptable safety risks.

Currently, commercially available self-developable indicator products in the blood product market are generally supplied in boxes containing cards of eight indicators with 200 indicators (25 cards) per box. The box needs to be opened for slitting and handling of the indicator cards and individual indicators.

Hence, there is a need in the healthcare industry for an indicator that provides a quick and clear indication of radiation dosage exposure when a syringe is subjected to a radiation, and enables a safer, more efficient workflow at reduced cost for healthcare providers.

### SUMMARY OF THE INVENTION

The present disclosure provides a flexible self-adhesive label comprising a radiation-sensitive indicator for a syringe. The label can be adhesively attached to at least one part of the syringe and does not overlap with the marking. The labels of this invention enable an improved fit and easier mounting onto 30 mL and 60 mL syringes that are irradiated and do not interfere with the markings on the syringe barrel. In a preferred embodiment, the labels contain a barcode and are efficiently packaged and dispensed from a roll.

One objective of the present disclosure relates to a flexible self-adhesive label comprising a radiation-sensitive indicator for a syringe. The syringe comprises a barrel part with a front part and a rear part, a tip at the front part of the barrel, a piston at the rear part of the barrel, and an exterior surface with a marking thereon. The label is adhesively attached to at least one part of the syringe and does not overlap with the marking.

Another objective of the present disclosure relates to a flexible self-adhesive label comprising a radiation indicator and a barcode for a syringe, The syringe comprises a barrel part with a front part and a rear part, a tip at the front part, a piston at the rear part, and an exterior surface with a marking thereon. The label is attached to at least one part of the syringe and does not overlap the marking. The barcode is printed on the label with information related to the label.

One more objective of the present disclosure relates to a method of labelling a syringe comprising a barrel part with a front part and a rear part, a tip at the front part, a piston at the rear part, and an exterior surface with a marking thereto, with a flexible self-adhesive label comprising a radiation indicator. The method comprises providing a flexible self-adhesive label having a release layer; removing the release layer from the label; and attaching the adhesive layer of the label to at least one part of the syringe that does not overlap the markings.

Another objective of the present disclosure relates to a kit comprising: a syringe comprising a barrel part with a front part and a rear part, having a tip at the front part of the barrel, having a piston at the rear part of the barrel, and having an exterior surface with a marking thereof; and a flexible self-adhesive label comprising a radiation indicator and optionally a barcode, adapted to be attached to the syringe.

One more objective of the present disclosure relates to a dispenser to house a roll of a flexible self-adhesive label comprising a radiation indicator; the dispenser comprising: a roll of a flexible self-adhesive label; a holding case; at least one opening to the holding case for dispensing the label; and optionally an outer top protective cover.

These and other objects of the present invention will become apparent in light of the following disclosure.

### BRIEF DESCRIPTION OF THE DRAWINGS

It is believed that the present invention will be better understood from the following description taken in conjunction with the accompanying drawings. The referenced drawings are not to be construed as limiting the scope of present invention. The referenced drawings for the label and dispenser orientations are not to be construed as limiting the scope of present invention.

FIG. 1 shows the flexible self-adhesive labels comprising radiation-sensitive indicators sensitive to Gamma and X-Rays;

FIG. 2 shows a syringe with which the label of FIG. 1 can be used;

FIG. 3 shows the syringes with flexible self-adhesive label in room temperature;

FIG. 4 shows the flexible self-adhesive label with a 2-D barcode;

FIG. 5 shows the flexible self-adhesive label assembly;

FIG. 6 shows the box/package of flexible self-adhesive label roll assembly; and

FIG. 7 shows the dispenser of flexible self-adhesive label.

It should be noted that these figures are diagrammatic and not drawn to scale. Relative dimensions and proportions of parts of these figures have been shown exaggerated or reduced in size, for the sake of clarity and convenience in the drawings.

### DETAILED DESCRIPTION OF THE INVENTION

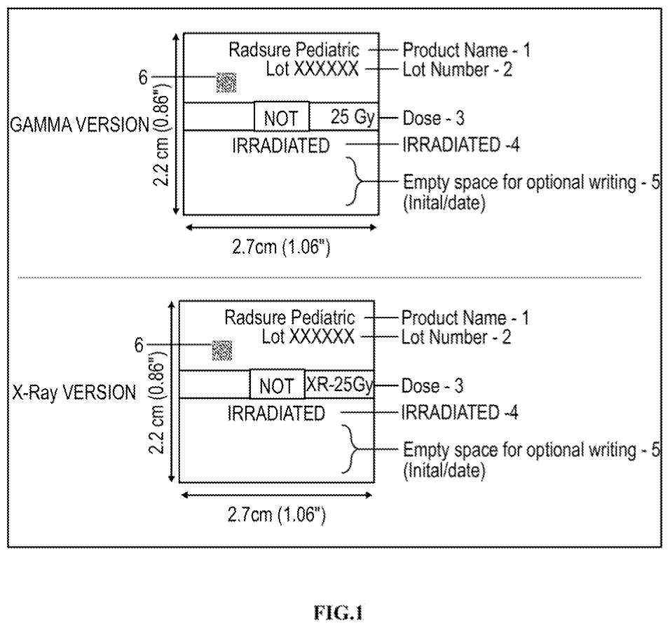

An exemplary embodiment of the invention will now be described with reference to FIGS. 1-7.

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed herein can be made and executed without undue experimentation in light of the present disclosure. While the articles and methods of the present disclosure have been described in terms of preferred embodiments, it will be apparent to those of ordinary skill in the art that variations can be applied to the articles and/or methods and in the steps or in the sequence of steps of the method(s) described herein without departing from the concept, spirit and scope of the present disclosure. All such similar substitutes and modifications apparent to those skilled in the art are deemed to be within the spirit, scope and concept of the present disclosure.

As utilized in accordance with the present disclosure, the following terms, unless otherwise indicated, shall be understood to have the following meanings.

The use of the word “a” or “an” when used in conjunction with the term “comprising” can mean “one,” but it is also consistent with the meaning of“one or more,” “at least one,” and “one or more than one.” The use of the term “or” is used to mean “and/or” unless explicitly indicated to refer to alternatives only if the alternatives are mutually exclusive, although the disclosure supports a definition that refers to only alternatives and “and/or.” Throughout this application, the term “about” is used to indicate that a value includes the inherent variation of error for the quantifying device, the method(s) being employed to determine the value, or the variation that exists among the study subjects.

References herein to “one embodiment,” or “one aspect” or “one version” or “one objective” or “another embodiment,” or “another aspect” or “another version” or “another objective” of the invention can include one or more of such embodiment, aspect, version or objective, unless the context clearly dictates otherwise.

The term “at least one” refers to one as well as any quantity more than one, including but not limited to, 1, 2, 3, 4, 5, 10, 15, 20, 30, 40, 50, 100, etc. The term “at least one” can extend up to 100 or 1000 or more depending on the term to which it is attached.

All percentages, parts, proportions, and ratios as used herein are by weight of the total composition, unless otherwise specified. All such weights as they pertain to listed ingredients are based on the active level and therefore do not include solvents or by-products that can be included in commercially available materials, unless otherwise specified.

All references to singular characteristics or limitations of the present invention shall include the corresponding plural characteristics or limitations, and vice-versa, unless otherwise specified or clearly implied to the contrary by the context in which the reference is made.

Numerical ranges as used herein are intended to include every number and subset of numbers contained within that range, whether specifically disclosed or not. Further, these numerical ranges should be construed as providing support for a claim directed to any number or subset of numbers in that range.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The terms “or combinations thereof” and “and/or combinations thereof” as used herein refer to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more items or terms, such as BB, AAA, AAB, BBC, AAABCCCC, CBBAAA, CABABB, and so forth. The skilled artisan will understand that typically there is no limit on the number of items or terms in any combination, unless otherwise apparent from the context.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that can vary depending upon the desired properties to be obtained in carrying out the invention.

The term “or combinations thereof”, “and combinations thereof”, and “combinations thereof” as used herein refers to all permutations and combinations of the listed items preceding the term.

The term “about” refers to a range of values+10% of a specified value. For example, the phrase “about 200” includes ±10% of 200, or from 180 to 220.

The term “polymerization” or “polymerizing” refers to methods for chemically reacting monomer compounds to form polymer chains. The polymer chain can be alternating, blocked, or random. The type of polymerization method can be selected from a wide variety of methods and include the following non-limiting examples: poly condensation, step growth polymerization, and free radical polymerization.

The term “macromolecule” refers to any large molecule, which includes polymers. The term “polymer” refers to a large molecule comprising one or more types of monomer residues (repeating units) connected by covalent chemical bonds. Non-limiting examples of polymers include homopolymers, and non-homopolymers such as copolymers, terpolymers, tetrapolymers and the higher analogues.

The term “syringe” refers to a device consisting of a glass, metal, or hard rubber tube, narrowed at its outlet, and fitted with either a piston or a rubber bulb for drawing in a quantity of fluid or for ejecting fluid in a stream, for example, through a needle, for cleaning wounds, injecting fluids into the body, etc., or any other suction device having similar features and purpose. The syringe can be a neonatal, pediatric, conventional, standard, or generic syringe.

The term “threshold dosage range” refers to the magnitude or intensity of radiation that must be exceeded for a certain reaction, phenomenon, result, or condition to occur or be manifested. Until the signal passes the threshold level there will be no change or reaction in the chemical compounds.

The term “QR Code” or “Quick Response Code” is a 2-D matrix code that conveys information by the arrangement of its dark and light elementary cells, also called “modules,” in columns and rows, i.e., in both the horizontal and vertical directions. Each dark or light module of a QR Code symbol—a specific instance of a code-represents a 0 or 1, thus making it machine intelligible. A QR code is detected by a 2-dimensional digital image sensor and then digitally analyzed by a programmed processor. The processor locates the three distinctive squares at the corners of the QR code image, using a smaller square (or multiple squares) near the fourth corner to normalize the image for size, orientation, and angle of viewing. The small dots throughout the QR code are then converted to binary numbers and validated with an error-correcting code.

The present disclosure is directed to a flexible self-adhesive label comprising a radiation-sensitive indicator for a syringe. The syringe comprises a barrel part with a front part and a rear part, having a tip at the front part of the barrel, a piston at the rear part of the barrel, and an exterior surface with a marking thereon. The label is attached to at least one part of the syringe and does not overlap with the marking.

According to one embodiment, syringe (100) comprises main body (11) having a portion for receiving label (12) and markings (13). The barrel (14) of the syringe, the main body has front end (15) and rear end (16), and tip (17) as a needle engaging extension at the front end of the main body. The tip has aperture (18) to load or release a fluid of the syringe with or without a needle. The syringe further comprises piston (19) at the rear end, wherein the piston comprises an opening adapted to receive the forward end of said piston rod. Disposed for slidable movement within barrel the piston is releasably coupled with barrel. The barrel further includes opposing side edges (20) that are straight and extend parallel to one another in a longitudinal direction to provide a grip to a user while using the syringe.

According to one of the embodiments, the barrel can be indicated, marked, shaped, or otherwise accustomed so as to indicate to a user a desired volume of a specific fluid to be drawn into the syringe. This can allow a syringe (which can be a neonatal, pediatric, conventional, standard, or generic syringe) to be marked in a consistent and reliable manner. The marking can enable the syringe to be customized for a specific purpose, in a way that reduces the likelihood of human error and enhances safety.

According to another embodiment, the tip of the syringe tends to fix the main body longitudinally on the barrel, thereby helping to ensure that the marking of the desired volume is provided at the correct location on the barrel. In general, the aperture need not be completely enclosed by the tip. For example, the aperture can comprise a slot or notch in the tip.

According to one more embodiment, the marking on the barrel comprises a numeric indication of a volume. The numeric indication of a volume is from 1 ml to 200 ml. In another embodiment, other possible ranges of numeric indication of a volume would include, but are not limited to, from about 0.1 ml about 10 ml; from about 1 ml to about 20 ml; from about 1 ml to about 30 ml; from about 1 ml to about 40 ml; from about 1 ml to about 50 ml; from about 1 ml to about 60 ml; from about 1 ml to about 70 ml; from about 1 ml to about 80 ml; from about 1 ml to about 90 ml; from about 1 ml to about 100 ml; from about 1 ml to about 110 ml; from about 1 ml to about 120 ml; from about 1 ml to about 130 ml; from about 1 ml to about 140 ml; from about 1 ml to about 150 ml; from about 1 ml to about 160 ml; from about 1 ml to about 170 ml; from about 1 ml to about 180 ml; from about 1 ml to about 19% ml; and from about 1 ml to about 200 ml.

According to one embodiment, a contiguous piece of material of the tip surrounds the opening so that the boundary of the opening defines a closed contour. The opening can be circular. The tip is preferably shorter than the main body, in a longitudinal direction of the marking. The tip is preferably narrower than the main body, in a transverse direction of the marking.

According to one more embodiment, the markings may comprise a mark/indication at a predetermined longitudinal position along the main body, which indicates the longitudinal position in the syringe-barrel to which the piston of the syringe should be pulled, in order to fill the syringe with the predefined volume of the fluid.

Further markings may be provided at different longitudinal positions, to indicate other different predefined volumes for other different predefined fluids. In some embodiments, the markings graphically indicate a longitudinal position and/or a longitudinal range of the barrel that corresponds to a safe dose of the predefined fluid. When the fluid is drawn into the syringe (for example, from an ampoule or vial), the piston is drawn to a position along the barrel that lies at the marked longitudinal position and/or lies within the graphically indicated range. The syringe is thereby known (and can be seen) to contain a safe dose of the fluid. Ranges and/or positions can also be indicated for different safe doses of other different fluids. A marked longitudinal position may indicate a nominal correct dose. This can be derived theoretically from experimental models or can be, for example, an average dose used by surgeons in the procedure. The average dose may be a median dose.

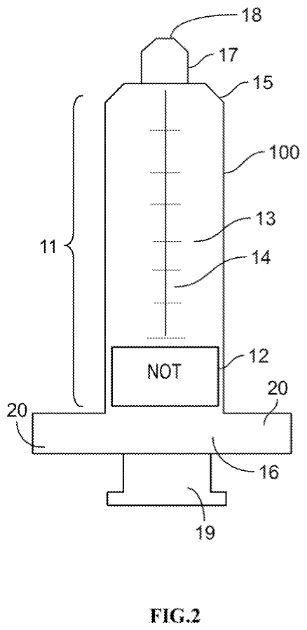

According to one more embodiment, a flexible self-adhesive label (12) is attached to at least one part of the exterior surface of the syringe, shown in FIG. 2. The label can be in any shape, or otherwise adapted so as to indicate whether the syringe is subjected to radiation or not. The label can be designed to meet each other ends once wrapped around the barrel of the syringe, thereby ensuring that they are applied to the correct diameter of syringe.

According one of the embodiments, the radiation-sensitive indicator can be adapted from a commercially available Rad-Sure™ blood irradiation indicator available from Ashland LLC that provides positive visual verification of irradiation at the minimum specified dose. Manufactured from Gafchromic™ film, the world's highest resolution dosimeter, Rad-Sure is the standard for blood irradiation indicators for over 25 years. The radiation-sensitive indicators can be sensitive to Gamma and/or X-Rays. Indicators sensitive to Gamma and X-Rays are depicted in FIG. 1. Gamma ray sensitivity is compatible with Cesium-137 or Cobalt-60 radiation sources and X-Ray sensitivity is compatible with x-ray irradiators that utilize x-rays generated from 160 kVp sources that are filtered through 0.38 mm of copper or 150 kVp sources that are filtered through 1 mm of aluminum. Radiation dosage indicators and their assembly are disclosed generally in U.S. Pat. Nos. 5,084,623 and 9,797,771.

According to one embodiment, the radiation dosage indicator is subjected to radiation at an energy level of at least from about 40 keV to about 10 MeV. In another embodiment, other possible ranges would include, but are not limited to, from about of 40 keV to about 50 keV; from about of 50 keV to about 60 keV; from about of 60 keV to about 70 keV; from about of 70 keV to about 80 keV; from about of 80 keV to about 90 keV; from about of 90 keV to about 100 keV; from about of 1 MeV to about 2 MeV; from about of 2 MeV to about 3 MeV; or from about of 3 MeV to about 4 MeV.

According to one embodiment, the radiation has a threshold dosage range from about 2500 rods to about 5000 rads. In another embodiment, other possible ranges would include, but are not limited to from about of 2500 rads to about 3000 rads; from about of 3000 rads to about 3500 rads; from about of 3500 rads to about 4000 rads; from about of 4000 rads to about 4500 rads; or from about of 4500 rads to about 5000 rads.

According one of the embodiments, the radiation has a threshold dosage range from about 10⁻⁸ J/cm² rads to about 10 J/cm². In another embodiment, other possible ranges would include, but are not limited to, from about of 10⁻⁸ J/cm² rads to about 10⁻⁷ J/cm²; from about 10⁻⁷ J/cm² rads to about 10⁻⁶ J/cm²; from about 10⁻⁶ J/cm² rads to about 10⁻⁵ J/cm2; from about 10⁻⁵ J/cm² rads to about 10⁻⁴ J/cm²; from about 10⁻⁴ J/cm² rads to about 10⁻³ J/cm²; from about 10⁻³ J/cm² rads to about 10⁻² J/cm²; from about 10⁻² J/cm2 rads to about 10⁻¹ J/cm²; from about 10⁻¹ J/cm² rods to about 1 J/cm²; from about 1 J/cm² rads to about 10¹ J/cm²; or from about 10² J/cm² rads to about 10³ J/cm².

According one of the embodiments, the radiation has a threshold dosage range from about 10⁻³ to 10⁸ rads. According one of the embodiments, the radiation has a threshold dosage range from about 10⁻³ to 10⁸ rads; from about 10⁻³ to 10⁻² rads; from about 10⁻² to 10⁻¹ rads; from about 10⁻¹ to 10 rads; from about 10 to 10¹ rads; from about 10¹ to 10² rads; from about 10² to 10³ rads; from about 10³ to 10⁴ rads; from about 10⁴ to 10⁵ rads; from about 10⁵ to 10⁶ rads; from about 10⁶ to 10⁷ rads; or from about 10⁷ to 10⁸ rads.

According to one of the embodiments, the radiation indicator comprises a radiation-sensitive polyacetylene compound. Non-limiting examples of polyacetylene compounds that are useful in present invention are disclosed in U.S. Pat. Nos. 5,137,964 and 7,445,880. It is specifically contemplated that any other comparable radiation sensitive material can be utilized in the practice of this invention.

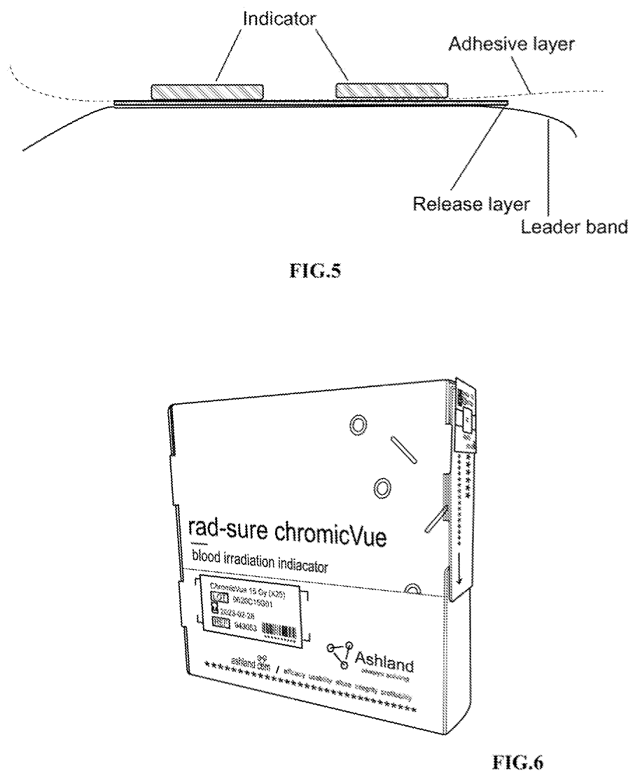

According to another embodiment, the flexible self-adhesive label further comprises a first adhesive layer, a leader band, a second adhesive layer, and a release layer, wherein first side of the adhesive layer, the leader band, or the release layer is attached to second side of the radiation-sensitive indicator, the adhesive layer, the leader band, or the release layer, shown in FIG. 5. The radiation-sensitive indicator is positioned on the first side of the adhesive layer. The second side of adhesive layer is attached to the first side of the release layer. Further, the second side of the release layer is attached to the first side of leader band.

According to one of the embodiments, the radiation dosage indicator is attached to the adhesive layer. Several useful adhesives for constructing the adhesive layer are available commercially, such as, for example, acrylic adhesives, acrylic urethane-based adhesives, and the like. The indicator can be fixed to double coated film product that comprises two adhesive layers. The double coated film product can be a plastic film with pressure sensitive adhesive applied to both sides.

According to another embodiment, the adhesive layer is positioned on one side of the release layer and the other side of the release layer is positioned on one side of leader band. The release layer has a mounting film for mounting radiation dosage indicator. The mounting film is available commercially, such as, for example MacTac PennaTrans PUV2100, IP2100, IB2104 and the like.

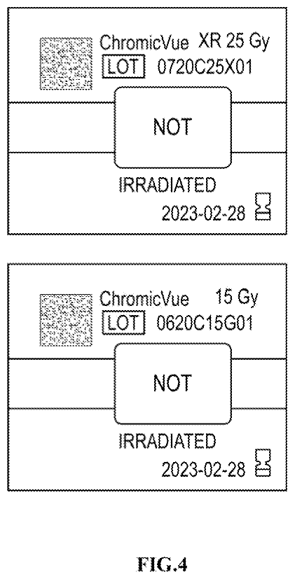

According to one of the embodiments, the flexible self-adhesive label comprises a radiation indicator and a barcode, shown in FIG. 4. The label can be attached to at least one part of the syringe and preferably does not overlap with the marking. The barcode is printed on the label and can include information related to the label.

According to another embodiment, the barcode is selected from the group consisting of radiation sensitive and radiation non-sensitive 2-D barcode.

According to another embodiment, the barcode is selected from the group consisting of a 2D codes, a QR Code (Quick Response Code), a micro QR code, a modified QR code, PDF-417, MaxiCode, Aztec Code, FAN barcodes and a datamatrix.

According to another embodiment, the barcode is selected from the group consisting of a 2D codes, a QR Code (Quick Response Code), a micro QR code, a modified QR code, PDF-417, MaxiCode, Aztec Code, EAN barcodes and a datamatrix.

According to one embodiment, the present disclosure relates to a method of labelling a syringe comprising a barrel part with a front part and a rear part, a tip at the front part, a piston at the rear part, and an exterior surface with a marking thereon. The above described flexible, self-adhesive label containing a radiation-sensitive indicator and a release layer is attached to at least one part of the syringe. The method comprises providing a flexible self-adhesive label; removing the release layer of the label; and attaching the adhesive layer of the label to at least one part of the syringe that does not overlap the marking.

According to one embodiment, there is provided a kit comprising: a syringe comprising a barrel part with a front part and a rear part, having a tip at the front part of the barrel, having a piston at the rear part of the barrel, and having an exterior surface with a marking thereof; and a flexible self-adhesive label, adapted to attach to the syringe as summarized above. The label is attached to at least one part of the syringe and does not overlap with the marking. The label can contain a barcode. According to another embodiment, the kit further comprises patient and/or healthcare worker educational material.

According to another embodiment, the kit comprises one or more self-adhesive labels for attaching to the syringe, shown in FIG. 6. The self-adhesive label can be one or more labels, a sheet of labels, a roll of radiation indicators, or a dispenser to house a roll of radiation indicators.

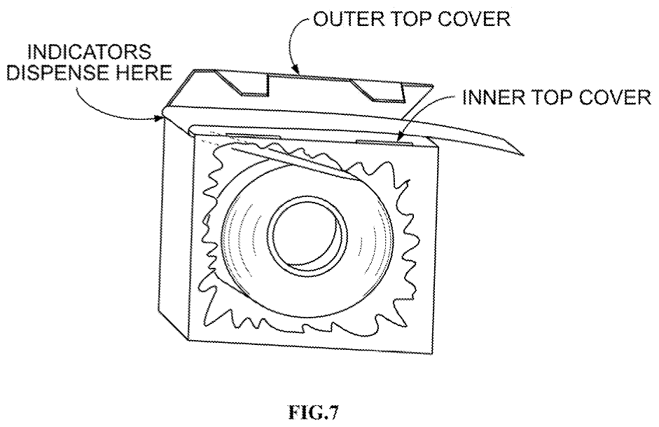

According to one more embodiment, a dispenser to house a roll of flexible self-adhesive labels comprises: a roll of a flexible self-adhesive label; a holding case; at least one opening to the holding case for dispensing the label; and optionally an outer top cover to the holding case, shown in FIG. 7. It is desirable that unused indicators not be exposed to light. Further, this packaging design enables a single indicator to be presented to the healthcare worker, reducing handling steps and eliminating slitting and handling of indicator cards.

According to another embodiment, the holding case is formed in the shape of a cube, cylindrical, half cylindrical or cuboid.

According to an alternative embodiment, the outer top cover protects the roll of labels as well as the individual labels.

According to another embodiment, the label is torn off from the roll prior to attachment to the syringe.

The following Examples are provided to illustrate certain features and advantages of various embodiments of the invention and should not be construed as limiting the scope thereof.

### EXAMPLES

Three syringes with flexible self-adhesive labels were prepared by adhesively attaching to each syringe the flexible self-adhesive labels of the present application. These three syringes were used for analyzing adhesion of the labels on the syringes in various temperatures storage conditions.

### Example 1: Adhesive Test

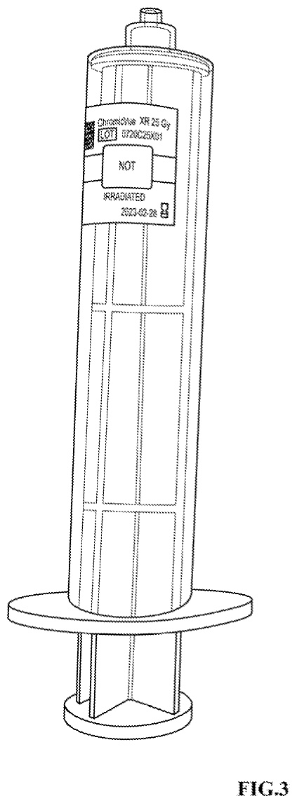

Indicators employing the adhesive were applied to the plastic syringe at room temperature (20 to 23° C.). The individual indicators were then held at room temperature (FIG. 3), refrigerator temperature (4 to 10° C.) and freezer temperature (−30 to −15° C.). The syringes were then visually inspected for adhesion to the syringe barrel (FIG. 3).

The indicators demonstrated the non-adhered performance for room temperature samples. The refrigerator and freezer samples continued to exhibit adhesion to the syringe. Hence, the indicators of the present application demonstrated the excellent adhesion required for the pediatric syringe application.

While this invention has been described in detail with reference to certain preferred embodiments, it should be appreciated that the present invention is not limited to those precise embodiments. Rather, in view of the present disclosure, many modifications and variations would present themselves to those skilled in the art without departing from the scope and spirit of this invention.

We claim:

1. A flexible self-adhesive label comprising a radiation-sensitive indicator for a syringe, said syringe comprising a barrel with a front part and a rear part, having a tip at the front part, a piston at the rear part, and an exterior surface with a marking thereon; wherein the label is attached to at least one part of the syringe and does not overlap with the marking.

2. The flexible self-adhesive label according to claim 1, further comprising an adhesive layer, a leader band, and a release layer;
wherein first side of the adhesive layer, the leader band, or the release layer is attached to second side of the radiation-sensitive indicator, the adhesive layer, the leader band, or the release layer.

3. The flexible self-adhesive label according to claim 2, wherein the radiation-sensitive indicator is positioned on the first side of the adhesive layer.

4. The flexible self-adhesive label according to claim 2, wherein the second side of adhesive layer is attached to the first side of the release layer.

5. The flexible self-adhesive label according to claim 2, wherein the second side of the release layer is attached to the first side of leader band.

6. The flexible self-adhesive label according to claim 1, wherein the radiation-sensitive indicator is sensitive to an energy level of at least from about 40 keV to 10 MeV.

7. The flexible self-adhesive label according to claim 1, wherein the radiation-sensitive indicator has a threshold sensitivity dosage range from about 2500 rads to about 5000 rads.

8. The flexible self-adhesive label according to claim 1, wherein the radiation has a threshold sensitivity dosage range from about 3,000 rads to 4,500 rads.

9. The flexible self-adhesive label according to claim 1, wherein the radiation has a threshold sensitivity dosage range from about 10⁻⁸ J/cm² to 10³ J/cm².

10. The flexible self-adhesive label according to claim 1, wherein the radiation has a threshold sensitivity dosage range from about 10⁻³ rads to 10⁸ rads.

11. The flexible self-adhesive label according to claim 1, wherein the marking on the barrel of the syringe comprises a numeric indication of volume.

12. The flexible self-adhesive label according to claim 11, wherein the numeric indication of volume is from about 10 ml to about 200 ml.

13. The flexible self-adhesive label according to claim 1, wherein the syringe is a pediatric or neonatal syringe.

14. The flexible self-adhesive label according to claim 1, wherein the tip has a circular opening.

15. The flexible self-adhesive label according to claim 1, wherein the piston comprises an axially movable ram.

16. The flexible self-adhesive label according to claim 1, wherein said radiation indicator comprises a chemical compound selected from the group consisting of a radiation-sensitive lithium polyacetylene compound and polyacetylene compound.

17. The flexible self-adhesive label according to claim 1, wherein the radiation indicator comprises a second label without a radiation sensitive element for notational purpose.

18. A flexible self-adhesive label comprising a radiation sensitive indicator and a barcode for a syringe, said syringe comprising a barrel part with a front part and a rear part, having a tip at the front part, a piston at the rear part, and an exterior surface with a marking thereon; wherein the label is attached to at least one part of the syringe and does not overlap the marking, and wherein the barcode is printed on the label with information.

19. The flexible self-adhesive label according to claim 18, wherein the barcode is selected from the group consisting of radiation sensitive and radiation non-sensitive 2-D barcode.

20. The flexible self-adhesive label according to claim 18, wherein the 2-D barcode is selected from the group consisting of a QR Code (Quick Response Code), a micro QR code, a modified QR code, a PDF-417, a MaxiCode, an Aztec Code, an EAN barcode and a datamatrix.

21. A method of labelling a syringe comprising a barrel part with a front part and a rear part, having a tip at the front part, a piston at the rear part, and an exterior surface with a marking thereon, wherein the label is attached to at least one part of the syringe and does not overlap with the marking, the method comprising:
providing the flexible self-adhesive label comprising a radiation indicator according to claim 2;
removing the release layer of the label; and
attaching the adhesive layer of the label to the syringe such that it does not overlap with said marking.

22. A kit comprising in combination:
a syringe comprising a barrel part with a front part and a rear part, having a tip at the front part, a piston at the rear part, and an exterior surface with a marking thereon; and
a flexible self-adhesive label comprising a radiation indicator according to claim 1, adapted to be attached to the syringe;
wherein the label is attachable to at least one part of the syringe that does not overlap with the marking.

23. A dispenser to house a roll of the flexible self-adhesive label comprising a radiation-sensitive indicator of claim 1; the dispenser comprising:
a roll of the flexible self-adhesive label of claim 1;
a holding case;
at least one opening to the holding case for dispensing a label; and optionally an outer top cover to the holding case.

24. The dispenser according to claim 23, wherein the holding case has the shape of a cube, cylindrical, half cylindrical, or cuboid.

25. The dispenser according to claim 23, wherein the outer top cover is present to protect the roll of labels.

26. The dispenser according to claim 23, wherein the label is torn off from the roll prior to attachment to the syringe.
